(19) United States
(12) Patent Application Publication
(10) Pub. No.: US 2009/0306748 A1
(43) Pub. Date: Dec. 10, 2009

(54) BODY THERMAL REGULATION/MEASUREMENT SYSTEM

(75) Inventors: Joseph C. MOLLENDORF, Amherst, NY (US); David PENDERGAST, Hamburg, NY (US)

Correspondence Address: NIXON PEABODY LLP - PATENT GROUP, 1100 CLINTON SQUARE, ROCHESTER, NY, 14604, US

(73) Assignee: The Research Foundation of State University of New York, Amherst, NY (US)

(21) Appl. No.: 12/477,721

(22) Filed: Jun. 3, 2009

Related U.S. Application Data
(60) Provisional application No. 61/058,665, filed on Jun. 4, 2008.

(51) Int. Cl. A61F 7/00

(52) U.S. Cl. 607/104

(57) ABSTRACT

The present invention relates to a body thermal regulation system. The system includes a fluid-circulating garment having a plurality of fluid-impervious compartments where each compartment is in contact with a different part of a body. The system also includes an expandable bladder to accommodate changes in pressure in the fluid circulation system. At least one heating/cooling unit is positioned to heat and/or cool fluid circulating to the fluid-impervious compartments in the garment. The system also includes a fluid circulation system positioned to circulate fluid between the heating/cooling unit and the plurality of fluid-impervious compartments in the garment. The present invention also relates to a method for measuring heat fluxes in different parts of a body. The method involves providing to a subject the body thermal regulation system according to the present invention. Then, the temperatures and flow rates of fluid entering and exiting each of the plurality of fluid-impervious compartments in the garment are measured, allowing heat fluxes in different parts of the body to be determined.

This application claims benefit of U.S. Provisional Patent Application Ser. No. 61/058,665, filed Jun. 4, 2008.

The subject matter of this application was made with support from the Office of Naval Research (Grant No. N00014-02-10278). The U.S. Government may have certain rights in this invention.

### FIELD OF THE INVENTION

The present invention relates to a body thermal regulation system. The present invention also relates to a method for measuring heat fluxes in different parts of a body.

### BACKGROUND OF THE INVENTION

The rigors associated with diving are many. Surviving and capably functioning in cold or hot water temperatures are problems as old as diving itself. Water conducts heat away and toward the human body about 25 times faster than in air, and the heat capacity of water is greater than that of air by more than 3,500 times. The significant difference between rates of heat loss or gain at similar temperatures in air and when immersed in cold or hot water creates a distinct difference in the physiological effects associated with the two. Immersion in extreme cold or hot water environments can induce serious physiological effects that are potentially dangerous for a diver and since most diving is done in cold or hot water, adequate diver thermal protection is needed.

Currently, divers rely on insulated suits to keep them warm. Unfortunately, insulation is not sufficient for cold temperatures and generally not used in hot temperatures. The lack of adequate diver thermal protection in cold or hot water environments is currently mission limiting, although on-surface systems can provide marginally sufficient protection. These systems work via the use of a utility umbilical cord physically connecting the diver to a surface located support system which supplies the diver with heating or cooling fluid. However, because of the need for the utility umbilical cord, maneuverability of the diver is severely limited, suggesting that there is a need for an expandable and portable thermal regulation system. Unfortunately, current portable systems are unsuitable for real world applications, being limited by power density requirements, complicated hardware, and insufficient real-time environmental and system controls.

In hot water environments, the problem of body thermal regulation is exacerbated because the diver cannot adequately rid his body of the inevitable heat produced by normal or accelerated metabolism. This is in contrast to cold water environments where metabolically-produced heat helps keep a diver warm.

Furthermore, in hot water, there is additional heat gain from the hot surrounding ambient.

In addition, although measuring body core temperature and skin temperature is commonplace, the measurement of thermal balance in selected zones of the body has not been done. Moreover, efforts to integrate these measurements with a model to study internal heat storage and distribution have not been made.

Similar suits are known in the art including U.S. Patent Publication No. 2006/0144557 to Koscheyev, et al. However, the fluid circulation systems incorporated in these suits can be susceptible to cavitation and fluid hammer and the problems associated with them (e.g., pump or impeller damage, or pressure surges), particularly if the fluid circulation is achieved using a positive displacement pump (e.g., a gear pump).

When a volume of liquid is subjected to a sufficiently low pressure it may rupture and form a cavity. This phenomenon is termed cavitation inception and may occur behind the blade of a rapidly rotating propeller. Vapor gases evaporate into the cavity from the surrounding medium. Such a low pressure cavitation bubble in a liquid will begin to collapse due to the higher pressure of the surrounding medium. As the bubble collapses, the pressure and temperature of the vapor within will increase. The bubble will eventually collapse, releasing a significant amount of energy. At the point of total collapse, the temperature of the vapor within the bubble may be several thousand Kelvin, and the pressure several hundred atmospheres.

Cavitation occurs in pumps, on propellers, or at restrictions in a flowing liquid. When the bubbles collapse, they typically cause very strong local shockwaves in the fluid. In devices such as propellers and pumps, cavitation causes a great deal of noise, damage to components, vibrations, and a loss of efficiency. The pitting caused by the collapse of cavities produces great wear on components and can dramatically shorten the lifetime of the propeller or pump.

Water hammer (or, more generally, fluid hammer) is a pressure surge or wave caused by the kinetic energy of a fluid in motion when it is forced to stop or change direction suddenly. It depends on the fluid compressibility where there are sudden changes in pressure. For example, if a valve is closed suddenly at an end of a pipeline system, a water hammer wave propagates in the pipe. Moving water in a pipe has kinetic energy proportional to the mass of the water in a given volume times the square of the velocity of the water.

The present invention is directed to overcoming these deficiencies in the art.

### SUMMARY OF THE INVENTION

The present invention relates to a body thermal regulation system. The system includes a fluid-circulating garment having a plurality of fluid-impervious compartments where each compartment is in contact with a different part of a body. At least one heating/cooling unit is positioned to heat and/or cool fluid circulating to the fluid-impervious compartments in the garment. The system also includes a fluid circulation system positioned to circulate fluid between the heating/cooling unit and the plurality of fluid-impervious compartments in the garment. The fluid circulation system includes at least one expandable bladder wherein said expandable bladder(s) accommodates pressure changes within said fluid circulation system.

Another aspect of the present invention relates to a method for measuring heat fluxes in different parts of a body. The method involves providing to a subject the body thermal regulation system according to the present invention described above. Then, the temperatures and flow rates of fluid entering and exiting each of the plurality of fluid-impervious compartments in the garment are measured. Finally, the heat fluxes in different parts of the body are determined.

The present invention discloses a total body thermal measurement system that also has the capability to alter and regulate the thermal status of a body. The body thermal regulation system of the present invention is capable of removing and delivering extra body heat independently from and to different zones of the body, respectively. This system has measurement and medical applications as well. Thus, the present invention also discloses a body thermal measurement system that is capable of determining the skin temperature and heat flux in different zones of a body, as well as the total body, allowing the estimation of deep body temperatures, distribution and storage of heat, convective heat transfer and core temperature using a thermal model to integrate the measured data.

### BRIEF DESCRIPTION OF THE DRAWINGS

FIG. 1 is a front cross-sectional view of a body thermal regulation system according to the present invention, specifically being used as a diver thermal protection package.

FIG. 2 is a top view of a body thermal regulation system according to the present invention.

FIG. 3 is a cross-sectional side view of the body thermal regulation system shown in FIG. 2.

FIG. 4 is an end view of the body thermal regulation system shown in FIG. 2.

FIG. 5 shows a schematic diagram of a body thermal regulation system according to the present invention.

FIG. 6 is a side view of a diver with a body thermal regulation system according to the present invention, which is capable of regulating the temperature of six different parts of a body, i.e., torso, head, hands, arms, legs, and feet.

FIG. 7 is a top view of a diver with a body thermal regulation system according to the present invention, which is capable of regulating the temperature of six different parts of a body, i.e., torso, head, hands, arms, legs, and feet.

FIG. 8 is an enlarged detail of FIG. 1., showing a front cross-sectional view of a body thermal regulation system including exemplary alternative locations for expandable bladder 800.

FIG. 9 shows a schematic diagram of a body thermal regulation system according to the present invention including exemplary alternative locations for expandable bladder(s).

FIG. 10 is a schematic of the associated typical local static pressures in the body thermal regulation system with (upper trace) and without (lower trace) an expandable bladder.

### DETAILED DESCRIPTION OF THE INVENTION

The present invention relates to a body thermal regulation system. The system includes a fluid-circulating garment comprised of a plurality of fluid-impervious compartments which can be in contact with different parts of the body. At least one heating and/or cooling unit is positioned to heat and/or cool fluid circulating to the fluid-impervious compartments of the garment. The fluid circulation system is positioned to circulate fluid between the heating and/or cooling unit and the plurality of fluid-impervious compartments in the garment. The fluid circulation system includes at least one expandable bladder to accommodate pressure changes within said fluid circulation system.

The expandable bladder may be a sac or bag-like structure designed to expand (increase its volume) or contract (decrease its volume) depending on changes in pressure within the fluid circulation system. The bladder may be made of rubber or other elastomeric material, or other suitable materials known by those in the art to have the desired properties, including imperviousness to fluids and the ability to expand and contract. The expandable bladder may be have one or more inlets or outlets and may be plumbed inline with the tubing of the fluid circulation system via a single inlet/outlet or via separate inlet(s) and outlet(s).

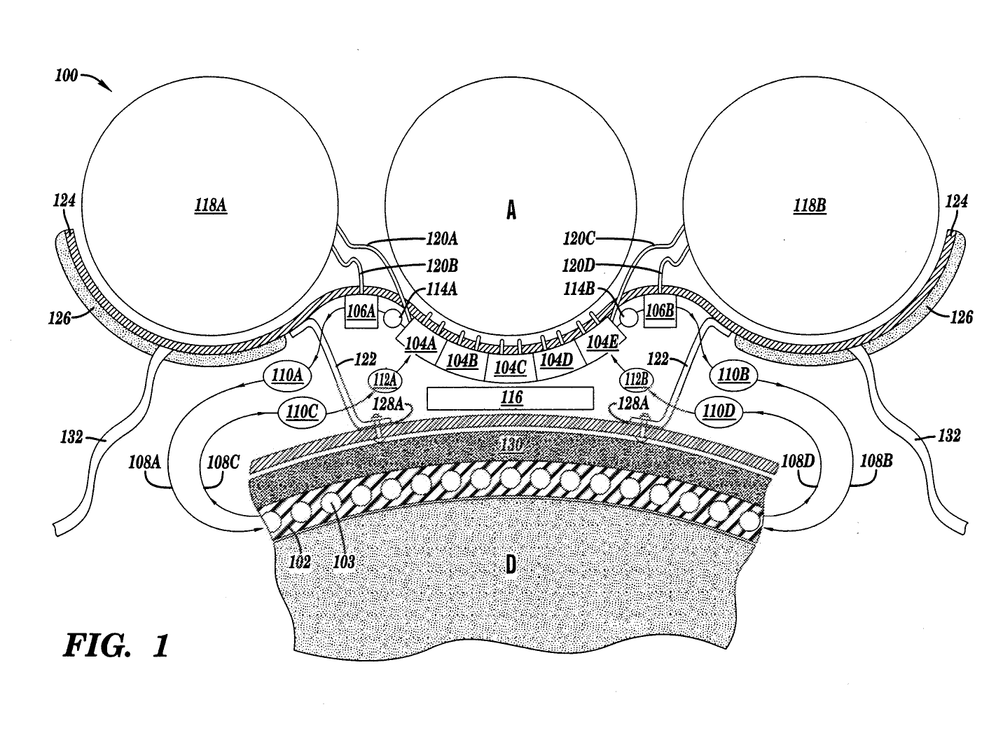

FIG. 1 shows a front cross-sectional view of a body thermal regulation system according to the present invention. Specifically, the system in FIG. 1 shows a thermal protection package for divers. Thus, FIG. 1 depicts a body thermal regulation system, generally referred to as reference numeral 100, used in conjunction with air tank A on diver's back D. In the embodiment shown in FIG. 1, body thermal regulation system 100 contains fluid circulating garment 102, heating/cooling units 104A-E, and pumps 106A-B. Fluid-circulating garment 102 having plurality of fluid-impervious compartments 103 can be a tube suit or a flat panel suit having flat channels. Heating/cooling units 104A-E can be arranged in series or in parallel for heating and/or cooling fluid circulating to the fluid-impervious compartments 103 in garment 102. Suitable examples of a heating/cooling unit include, but are not limited to a thermoelectric unit, an electrical resistance heater, a heat pump, a vapor compression refrigerator, and a thermodynamic refrigerator. Preferably, the heating/cooling unit is a thermoelectric unit. Pumps 106A-B are positioned to draw fluid from heating/cooling units 104A-E and expel the heated or cooled fluid into fluid-impervious compartments 103 in garment 102 through fluid transporting tubes 108A-B past quick disconnects 110A-B. Fluid-impervious compartments 103 of garment 102 are connected to another set of fluid transporting tubes 108C-D which transport fluid via quick disconnects 110C-D to heating/cooling units 104A-E.

Body thermal regulation system 100 can further contain inlet manifolds 112A-B positioned to receive fluid from plurality of fluid-impervious compartments 103 in garment 102 and outlet manifolds 114A-B positioned to receive fluid from heating/cooling units 104A-E.

Body thermal regulation system 100 further contains controller 116, which is in electrical communication with heating/cooling units 104A-E and the fluid circulation system. The body thermal regulation system of the present system further contains at least one power source for providing power to the heating/cooling units and the fluid circulation system. The power source can be a fuel cell or a battery. Fuel cells which use hydrogen and oxygen to produce electricity, heat, and water can be used as the power source. In addition, fuel cells possessing high energy densities at low weights, such as portable underwater aluminum-oxygen fuel cells, can be used in conjunction with the body thermal regulation system of the present invention. When fuel cells are employed, the use of a waste heat recovery system (e.g., heat exchangers) that utilizes the unused heat from the fuel cell to heat the diver is particularly appropriate in cold water ambients. However, recent improvements, and projected future improvements, in battery technology allow the use of batteries having high power density and peak power as the power source for the body thermal regulation system of the present invention. FIG. 1 depicts such a system, where body thermal regulation system 100 contains batteries 118A-B as the power source. Examples of batteries include, but are not limited to, lithium polymer batteries, lead/acid rechargeable batteries, and lithium solid state batteries. The choice of the battery will be determined by the needs of the specific mission or task to be completed using the body thermal regulation system of the present invention. Batteries 118A-B provide power to heating/cooling units 104A-E and pumps 106A-B through electrical connections 120A-D.

Body thermal regulation system 100 further contains housing 122 that encloses heating/cooling units 104A-E and pumps 106A-B, as well as other components of the system. In other embodiments, batteries 118A-B can be positioned within housing 122. Housing 122 can also form platform 124 for the external attachment of batteries 118A-B and air tank A. Housing 122 can be composed of thermal conducting materials. Further, certain parts of housing 122, such as the part forming platform 124, can be made of high thermal conducting material capable of collecting or dissipating excess heat 126. Examples of high thermal conducting material include, but are not limited to, copper, aluminum, and other metals and non-metals with high thermal conductivities. Gaskets 128A-B seal housing 122 from the external environment. In addition, body thermal regulation system 100 can further contain insulation layer 130 positioned outside of and adjacent to or as a part of fluid-circulating garment 102. Suitable insulations include insulation that can be used in conjunction with a fluid-circulating garment and can partially insulate the body from the environment, particularly in water, as the insulation described in U.S. patent application Ser. No. 10/645,726 to Mollendorf et al. The system of the present invention can also be used with currently available dry and wet suit designs, where fluid-circulating garment 102 would be positioned between the dry or wet suit and the diver's body. Body thermal regulation system 100 can also further contain harness straps 132 attached to housing 122 which allows the system of the present invention to be attached to a body.

The inside of housing 122 can be dry or, alternatively, housing 122 can be filled with a non-electrical conducting fluid, such as inert perfluorocarbons, E5 Freon, fluorinated polyoxy propylene, and other non-electrically conducting fluids. Filling the housing with an incompressible fluid, or alternatively pressurized gas, can be advantageous in the case of high pressure underwater environments and for heat dissipation. If housing 122 is filled with fluid that is electrolytic, then each of the components within the housing must be individually sealed to make them impervious to the fluid.

The body thermal regulating system of the present invention may be portable or non-portable, i.e. stationary or fixed. In the case of a portable system used for heating or cooling a diver, the system would be attached to the back of the diver, allowing free movement of the diver in the water environment. In the case of a non-portable system used for heating or cooling a diver, the system would be based on shore, on a boat, or on any other platform suitable for diving applications, where the diver would be connected to the system via a utility umbilical cord that transports heated or cooled fluid and/or electrical power to the diver as necessary. If the system is used for heating or cooling an individual in non-water environments, the system can be stationed on any suitable platform depending on the mission or task to be completed with the system of the present invention.

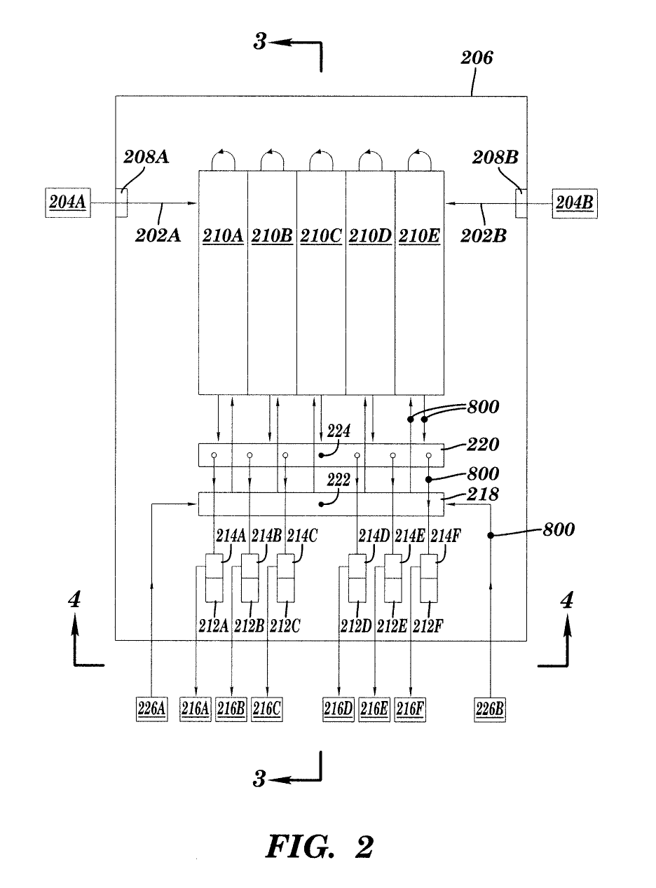

FIG. 2 depicts a top view of a body thermal regulation system according to the present invention. Electrical connections 202A-B from power sources 204A-B, such as batteries or fuel cells, enter housing 206 through waterproof penetration seals 208A-B and connects to heating/cooling units 210A-E. Power sources 204A-B also provide power to motors 212A-F which run pumps 214A-F associated to them. FIG. 2 depicts four alternate exemplary locations for expandable bladder 800. One or more expandable bladder(s) can be located in these positions or other positions within the fluid circulation system. FIG. 2 depicts an embodiment where the fluid circulation system includes pumps 214A-F, each having motors 212A-F, where pumps 214A-F are positioned to draw fluid from heating/cooling units 210A-E and expel, via six quick disconnects 216A-F, the heated or cooled fluid into each of the plurality of fluid-impervious compartments in the fluid-circulating garment. The system shown in FIG. 2 also contains inlet manifold 218 positioned to receive fluid, via quick disconnects 226A-B, from the plurality of fluid-impervious compartments in the fluid-circulating garment, as well as outlet manifold 220 positioned to receive fluid from heating/cooling units 210A-E. In addition, as shown in FIG. 2, inlet manifold 218 can contain temperature sensor 222 positioned for measuring the average or “cup-mixing” temperature of fluid drawn from the plurality of fluid-impervious compartments in the fluid-circulating garment, while outlet manifold 220 can contain temperature sensor 224 positioned for measuring the average or “cup-mixing” temperature of heated or cooled fluid from heating/cooling units 210A-E. In another embodiment, both inlet manifold 218 and outlet manifold 220 can contain a plurality of temperature sensors positioned for measuring the local temperature of each fluid entering and exiting each of the plurality of fluid-impervious compartments in the garment. In yet another embodiment, a plurality of temperature sensors can be positioned to measure the temperature of the external environment surrounding the body.

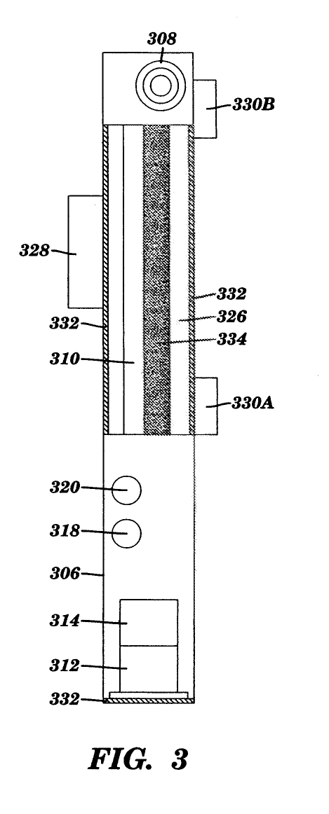

FIG. 3 depicts a cross-sectional side view of the body thermal regulation system shown in FIG. 2, showing housing 306 containing heating/cooling unit 310, controller 326, motor 312, pump 314, inlet manifold 318, and outlet manifold 320. Also depicted in FIG. 3 is external cradle 328 for holding the power source (not shown) and the air tank (not shown), bosses 330A-B for attaching housing 306 to a backpack (not shown) to be worn by an individual, and waterproof penetration seal 308 through which the electrical wires enter housing 306. Certain regions of housing 332 that are adjacent to and in contact with components within the housing which produce excess heat waste, such as motor 312, controller 326, and heating/cooling unit 310, can be made of high thermal conducting material capable of collecting or dissipating excess heat. FIG. 3 also shows insulation layer 334 positioned between controller 326, and heating/cooling unit 3 10. Electrical connections from the power source enter housing 306 through waterproof penetration seal 308 and connect to heating/cooling unit 310, inlet manifold 318, outlet manifold 320, and controller 326 (connections not shown).

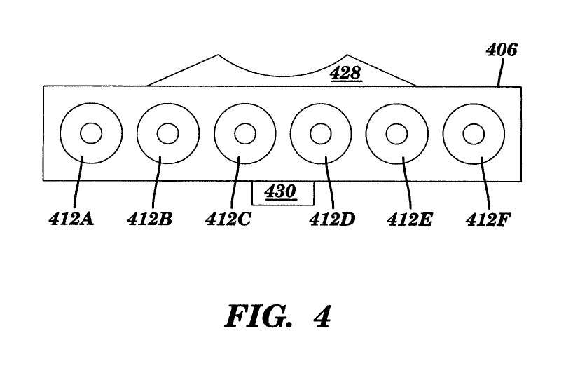

FIG. 4 shows an end view of the body thermal regulation system shown in FIG. 2, showing housing 406 containing motors 412A-F. Also depicted in FIG. 4 is external cradle 428 for holding the power source (not shown) and the air tank (not shown). External cradle 428 can also act as a heat sink, allowing for the dissipation of waste heat from the system and may be composed of high thermal conducting material as described above. Boss 430 allows for attachment of housing 406 to a backpack (not shown) to be worn by an individual.

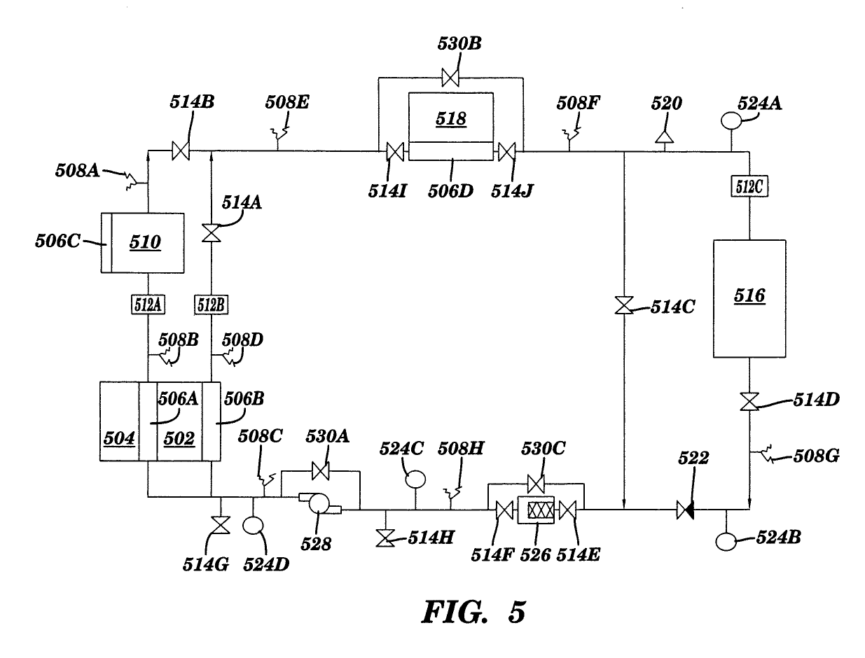

FIG. 5 shows a schematic diagram of a body thermal regulation system according to the present invention. FIG. 5 shows one possible combination of components for the system. However, depending on the mission or task to be completed and the power source used, the combination of components used in the body thermal regulation system may be varied. Heating/cooling unit 502 is controlled by attached controller 504 and heats or cools fluid flowing through it via heat exchangers 506A-B. In one embodiment, the heating/cooling unit is a thermoelectric cooler which uses the Peltier effect to heat or cool circulating fluid depending on its polarity.

In other embodiments, temperature sensors 508A-H are positioned throughout the system in order to regulate and measure the temperature of fluid within the system. Temperature sensors 508A-B are positioned to measure the temperature difference along power source 510. Temperature sensors 508B-C are positioned to measure the temperature along the “cold” side of heating/cooling unit 502, while temperature sensors 508C-D are positioned to measure the temperature difference along the “hot” side of heating/cooling unit 502. Temperature sensor 508E is positioned to measure the temperature of fluid in the system after the mixing of the “hot” and “cold” sides. In addition, heat exchanger 506C can be attached to power source 510, as a means of recycling waste heat from power source 510.

Flowmeters can also be positioned throughout the thermal regulation system of the present invention. Flowmeter 512A is positioned to measure the fluid flow rate in the “cold” side line of heating/cooling unit 502, while flowmeter 512B is positioned to measure the fluid flow rate in the “hot” side line of heating/cooling unit 502. In addition, flow regulators 514A-B can be positioned to regulate the flow in these lines of the system.

The system can also contain temperature sensor 508F positioned to measure the inlet temperature of garment 516. This temperature controls how much heat is transferred to or from phase change material 518, when phase change material is employed in the system of the present invention. In one embodiment, the phase change material can be designed to absorb or release heat at about 40° C., melting or solidifying based on the circumstances and transferring heat to the system through heat exchanger 506D.

If the pressure in the system becomes too high, pressure release valve 520 is positioned to shut off the flow of fluid into garment 516. Since the flow rate of fluid within garment 516 may be significantly less than the flow rate of fluid through power source 510, flowmeter 512C is positioned to measure the flow rate of fluid into garment 516, while flow regulators 514C-D are positioned to balance the flow rates. In addition, temperature sensors 508F-G are positioned to measure the temperature difference along garment 516.

Fluid entering garment 516 will perfuse through different zones at various temperatures, heating or cooling a body depending on the surrounding temperature. Backflow valve 522 prevents fluid from flowing back into garment 516 after it has already passed through garment 516. In addition, pressure instruments 524A-B are positioned to measure the pressure difference along garment 516, while temperature sensor 508H is positioned to measure the mixing temperature of fluid coming from garment 516 and an adjacent line.

The system of the present invention can also further contain filter 526 positioned to remove fibrous materials floating through the system. The suction pressure of pump 528 is measured by pressure instruments 524C-D, while temperature sensors 508H and 508C are positioned to measure the temperature difference along the pump. Bypass valve 530A relieves high pressure within pump 528, while bypass valves 530B-C are maintenance valves which can be opened when adjacent components are removed during specific functions. Shutoff valves 514E-F can be employed to force water to flow outside of filter 526, while shutoff valves 514G-H can be employed to force water to flow outside of pump 528. In addition, shutoff valves 5141-J can be employed to force water outside of phase change material 518 as needed.

In operation, the regulation of body temperature of an individual using a body thermal regulation system of the present invention, as shown in FIGS. 1-5 can be carried out as follows. The fluid circulation system is first charged with a fluid, such as water, oil, or any other heat transferring fluid, which acts as the heat or cold transfer medium. Such fluids would have appropriate thermal and flow properties to accomplish the effective transfer of heat and would preferably be non-toxic and non-corrosive. The fluid is heated or cooled by heating/cooling units, which may be connected in parallel or series. FIG. 1 depicts a specific embodiment, where five heating/cooling units 104A-E are positioned to receive fluid from inlet manifolds 112A-B, which collectively receive fluid from plurality of fluid-impervious compartments 103 in garment 102. The arrowed lines shown in FIG. 1 depict the flow of fluid in the system. After the fluid is heated or cooled, it leaves heating/cooling units 104A-E and enters outlet manifolds 114A-B. FIG. 1 depicts a single outlet manifold on each side of the heating/cooling units; however, there can be any number of outlet manifolds present. For example, the number of the outlet manifolds can be the same as the number of different compartments in contact with different parts of the body in the fluid-circulating garment. In addition, each of the at least one heating/cooling unit can be positioned to individually heat and/or cool fluid circulating to each of the plurality of fluid-impervious compartments in the garment. Next, the fluid leaves outlet manifolds 114A-B and enters pumps 106A-B. FIG. 1 depicts a single pump on each side of the heating/cooling units; however, there can be any number of pumps present. For example, the number of pumps can be the same as the number of different compartments in contact with different parts of the body in the fluid-circulating garment, where each of the plurality of fluid pumps are positioned to individually draw fluid from each of the at least one heating/cooling unit and expel the heated or cooled fluid into each of the plurality of fluid-impervious compartments in the garment. The fluid leaves pumps 106A-B through fluid transporting tubes 108A-B, exits housing 122 and enters plurality of fluid-impervious compartments 103 in garment 102 through quick disconnects 110A-B. The directional flow of fluid within the system will be the same whether the system is employed in cold or hot environments. In certain embodiments, the fluid pumps can be positive displacement pumps.

Fluid from plurality of fluid-impervious compartments 103 of garment 102 exits through another set of fluid transporting tubes 108C-D and, via quick disconnects 110C-D, enters inlet manifolds 112A-B. FIG. 1 depicts a single inlet manifold on each side of the heating/cooling units; however, there can be any number of inlet manifolds present. For example, the number of inlet manifolds can be the same as the number of different compartments in contact with different parts of the body in the fluid-circulating garment. The fluid then returns to heating/cooling units 104A-E to be heated or cooled. For diving applications, the temperature of the fluid within the system is typically maintained between about 22° C. to 39° C., although it can be maintained at any other temperature depending on the application.

Temperature sensors such as the ones 222, 224, shown in FIG. 2, positioned inside inlet manifolds 112A-B, and outlet manifolds 114A-B, send temperature data to controller 116. Controller 116 then directs heating/cooling units 104A-E to either heat or cool the fluid, as needed.

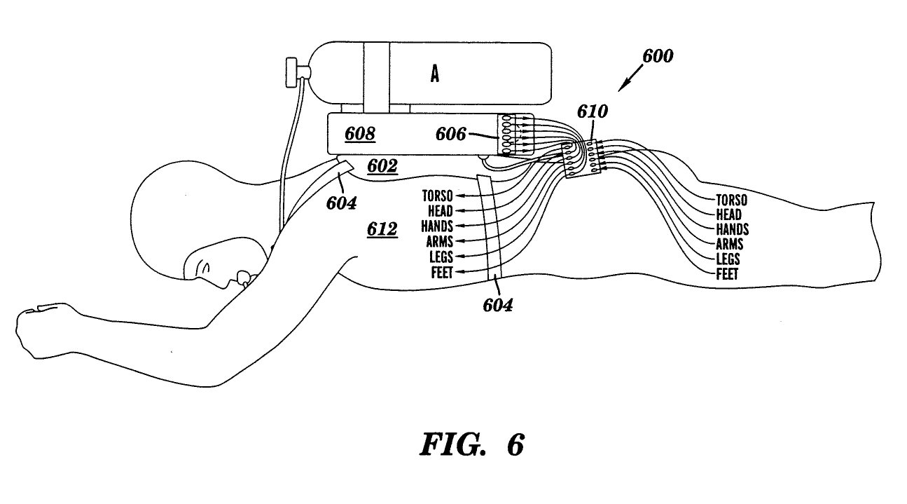

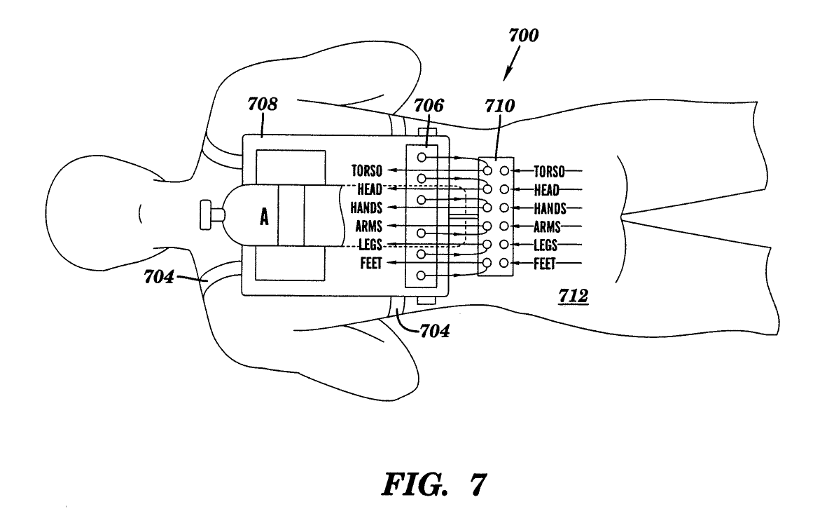

In one embodiment, the body thermal regulation system of the present invention can be set up so that the number of pumps, motors, inlet quick disconnects, and outlet quick disconnects employed directly coincides with the number of different compartments in the fluid-circulating garment, thereby individually regulating the temperature of different parts of the body. For example, the system can have multiple heating/cooling units and multiple pumps to individually heat or cool and pump fluid into different compartments in the fluid-circulating garment. FIGS. 6 and 7 show the side and top views, respectively, of a diver with such a body thermal regulation system 600, 700, which is capable of regulating the temperature of six different parts of a body, i.e., torso, head, hands, arms, legs, and feet. Arrowed lines indicate the direction of fluid flow. In another embodiment, external straps 604, 704 attach backpack 602 with body thermal regulation system 600, 700 and airtank A to the diver. In another embodiment, fluid leaves outlet manifold 606, 706 inside housing 608, 708 and enters external manifold 610, 710, which then flows into the six fluid-impervious compartments in garment 612, 712 in contact with six different parts of the body. The fluid circulates through the six fluid-impervious compartments in garment 612, 712, returns to external manifold 610, 710, flows through heating/cooling units (not shown), then returns to outlet manifold 606, 706 inside housing 608, 708. In other embodiments without the external manifolds, the fluid from the outlet manifold inside the housing would flow directly to the different fluid-impervious compartments in the fluid-circulating garment. In other embodiments of the present invention, any number of different fluid-impervious compartments in contact with different parts of a body can be present.

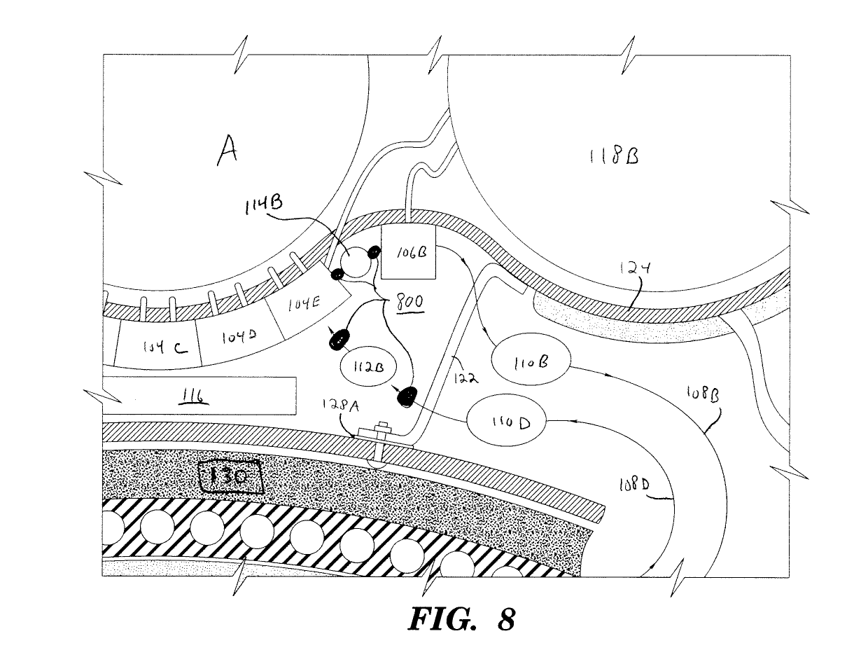

FIG. 8 shows alternate exemplary locations for expandable bladder 800 within housing 122. One or more expandable bladder(s) can be located in these positions or other positions within the fluid circulation system. Locating the expandable bladder(s) is not critical, since its purpose is to accommodate pressure changes within the fluid circulation system. However, locating expandable bladder(s) within housing 122 near body thermal regulation system 100 may be desired in terms of minimizing tube lengths and/or protecting the bladder(s) from damage.

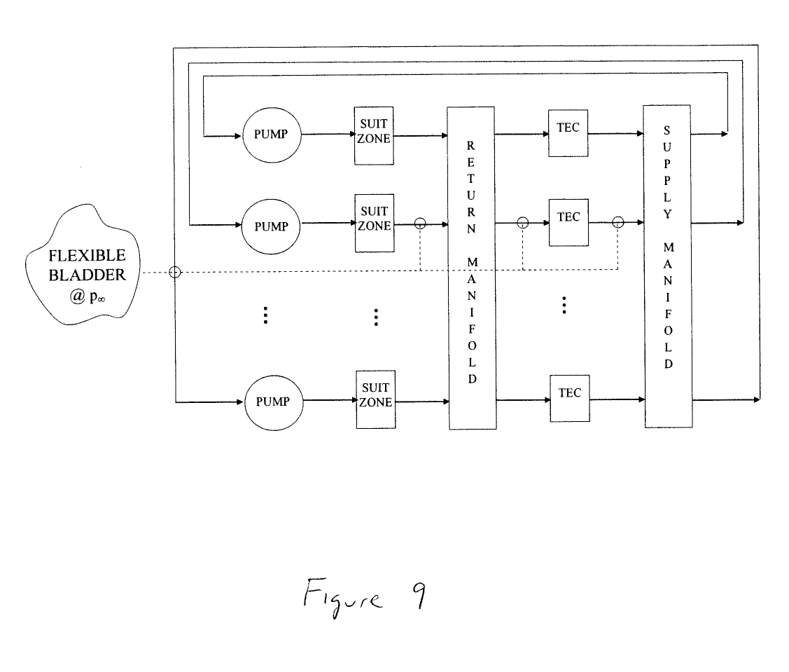

There are potential problems (e.g. water hammer and cavitation) associated with any closed-loop pumped system that are consequences of the characteristics of: the pump, the fluid, and the plumbing. Among these characteristics are the inevitable elasticity of the fluid conduits (pipes, tubing, and lines) and the essentially incompressible nature of liquids. For example, consider an incompressible fluid (such as water), a positive displacement pump (such as a gear pump), and flexible plastic tubing. A schematic of the body thermal regulation system plumbing is shown at FIG. 9. It can be seen that the pump(s) (e.g. pump 214) supply the suit zone(s) with water that has been thermally conditioned by the TEC (thermoelectric cooler), or heating and/or cooling unit(s) (e.g. heating/cooling unit 210) via the return (inlet) and supply (outlet) manifolds (e.g. inlet manifold 218 and outlet manifold 220). The system is typically filled with water at normal atmospheric pressure, and then closed. In a configuration (without the expandable (flexible) bladder 800), bubble formation, pump vapor-lock and drastically reduced flow (resulting in system failure) has been observed. This problem is eliminated by the incorporation of a flexible fluid-filled expandable bladder 800 that was plumbed-in, for example, between the suit and the return manifold. It is noted that the bladder could alternatively be plumbed-in, variously, via the dashed lines at connection points indicated by small circles of FIG. 9. The incorporation of this expandable bladder essentially eliminates bubble formation and results in proper flow and system performance.

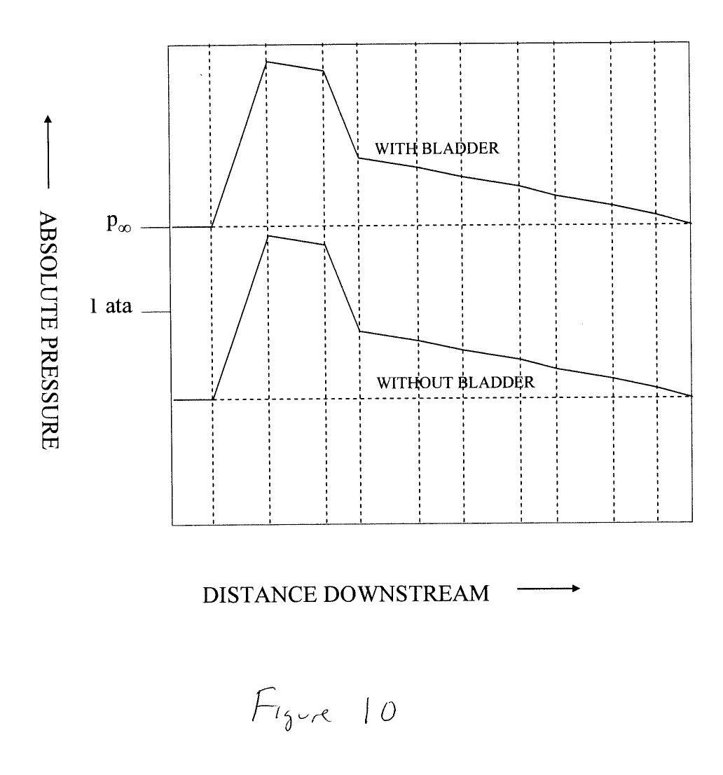

A schematic of the associated typical local static pressures in the body thermal regulation system is shown in FIG. 10 with (upper trace) and without (lower trace) the incorporation of the expandable bladder 800. It can be seen that without the bladder, the system pressure rises above atmospheric pressure (1 atm) as a result of the pressure head added by the pump, and then decreases below 1 atm as a result of the pressure drops in the system. The majority of the pressure drop occurs through the tube suit zone(s). It is noted that the total pressure drop could be typically more than 1 atm at flow rates of about 1000 liters per minute. It is well known that pump inlet pressures below lata are likely to result in cavitation (i.e. water vapor bubbles) as well as the formation of air bubbles, as dissolved air is released from solution. These bubbles cause vapor-lock in the pumps, essentially stop flow, and will eventually destroy the pumps. The incorporation of an expandable bladder raises the pressure level at the pump inlet to essentially that of the ambient pressure, which is typically at or above 1 atm. This inhibits the formation of air or water vapor bubbles.

The present invention also relates to a method for measuring heat fluxes in different parts of a body. The method involves providing to a subject the body thermal regulation system of the present invention described above. Then, the temperatures and flow rates of fluid entering and exiting each of the plurality of fluid-impervious compartments in the garment are measured. Finally, the heat fluxes in different parts of the body are determined. The overall principle of this method is to utilize the above-described body thermal regulation system to measure heat fluxes in multiple body zones and, hence the total body. In one embodiment, each compartment of the fluid-circulating garment has fluid pumped through it (typically at a flow rate from about 0.3 l/min up to about 1.6 l/min) by a dedicated pump located inside a housing. By measuring the temperature at the inlet and outlet to and from the garment, as well as the water flow, the heat flux can be calculated for each of the different zones of the body. By design, the inlet to the fluid-circulating garment can exit the housing from a manifold, and the outlet from the garment can enter the housing from another manifold. By measuring the temperature differential between the two manifolds, the total body heat flux can be determined. Interfaced between the two manifolds are parallel arrangements of a plurality of heating/cooling units, controlled by an integrated controller that adjusts the duty cycle of the heating/cooling units and heats or cools the fluid circulating through them. The power requirement of the heating/cooling units needed to maintain the body's skin and deep core temperatures is an additional measure of total body heat balance. The system of the present invention can thus serve as a total body calorimeter.

In parallel with the system measurements of the present invention, temperature and heat flux measurements can also be made directly from the body without the need to measure the temperature and flow of fluid using the thermal measurement system of the present invention. Thus, thermocouples and heat flow disks can be attached to the different zones of the fluid-circulating garment both on the body and insulation sides of the garment. The elasticity of the fluid-circulating garment material can act to press the thermocouples and heat flow disks against the skin of an individual's body. In addition, an ingestible capsule can be swallowed by the subject which is used to measure the core temperature and the heart rate of the individual. Using these data, temperatures and heat fluxes for different zones of the body can be measured and then integrated to estimate a body's total thermal status.

The data from the system's and body's measurements can be recorded on computer via analog/digital converters and stored for subsequent analysis. A computer model, based on quantitative physiology measurements taken during thermal stress experiments, can then be used to calculate the thermal status of the different zones of a body as well as the total body. Thus, the combination of the computer model and the data from the system's and body's measurement can be used to calculate regional temperatures within each of the different zones of a body, as well as body core temperature.

There are no comparable systems, which are currently available that perform the tasks that the system of the present invention is capable of performing. It is a unique combination of thermal measures from the body and the mechanical system and, when combined with known physiological data, has the power to address various issues regarding thermal regulation and measurement of a body. The system of the present invention has capabilities beyond those currently available. For example, it will not only provide thermal protection, but will also do total body calorimetry of an individual performing many different dynamic activities. Another advantage is that the system has the potential to work in both air and water environments with a wide range of thermal parameters and at altitude and underwater depth. The system has the advantage of permitting measurements while the person is thermally comfortable in many environments. The use of the computer model noted above will not only allow estimates of deep temperatures, temperature distribution and physiological control, but also has the capability to predict the physiological response to environmental stress in unusual environments.

Furthermore, the present system also has novel occupational and environmental safety applications. Manufacturers, firefighters, and individuals working with hazardous materials can be exposed to elevated environmental temperatures. The system of the present invention can be used to quantitatively assess the thermal stresses experienced by these workers. This information can be used to develop mechanisms for protecting these individuals from thermal stresses when working in these environments. The thermal states of professional divers working on construction, rescue, or military operations can also be evaluated with this system and lead to more effective thermal regulation for these workers performing these operations. Indeed, the system of the present invention has great potential to aid in the development of increased safety standards for workers in many fields and can in fact actually protect workers from work-related environmental thermal stresses. The system has potential medical applications as well, since it can be used for the measurement of thermal stresses on a patient undergoing treatment and for protecting these patients from treatment and illness-related thermal stresses, including stresses experienced by multiple sclerosis patients and post-operation surgical patients. In addition, when the system of the present invention is used in its portable mode, it can provide cooling or heating to patients and medical personnel. In particular, patients with thermal sensitivities, such as patients with multiple sclerosis, could use this system allowing them the ability to perform their daily activities in a safe and comfortable manner.

The foregoing description of the specific embodiments will so fully reveal the general nature of the present invention that others skilled in the art can, by applying current knowledge, readily modify or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of forms without departing from the invention.

What is claimed:

1. A body thermal regulation system comprising:
a fluid-circulating garment comprising a plurality of fluid-impervious compartments, each compartment being in contact with a different part of a body; and
at least one heating/cooling unit positioned to heat and/or cool fluid circulating to the fluid-impervious compartments in said garment; and
a fluid circulation system positioned to circulate fluid between the heating/cooling unit and the plurality of fluid-impervious compartments in said garment, wherein said fluid circulation system comprises at least one expandable bladder to accommodate pressure changes within said fluid circulation system.

2. The body thermal regulation system according to claim 1, further comprising:
a controller in electrical communication with the at least one heating/cooling unit and the fluid circulation system, whereby the at least one heating/cooling unit and the fluid circulation system can be controlled to heat or cool fluid circulating through the heating/cooling unit and the plurality of fluid-impervious compartments in said garment to regulate temperatures of different parts of the body.

3. The body thermal regulation system according to claim 1, further comprising:
at least one power source for providing power to the at least one heating/cooling unit and the fluid circulation system, wherein the power source, the heating/cooling unit, and the fluid circulation system are attached to or adjacent to said garment.

4. The body thermal regulation system according to claim 3, wherein the at least one power source is a fuel cell or a battery.

5. The body thermal regulation system according to claim 1, further comprising:
a housing enclosing the at least one heating/cooling unit and the fluid circulation system.

6. The body thermal regulation system according to claim 5, wherein said expandable bladder is located within said housing.

7. The body thermal regulation system according to claim 5, wherein said housing comprises a material capable of collecting or dissipating heat.

8. The body thermal regulation system according to claim 1, wherein said heating/cooling unit is selected from the group consisting of a thermoelectric unit, an electrical resistance heater, a heat pump, a vapor compression refrigerator, and a thermodynamic refrigerator.

9. The body thermal regulation system according to claim 8, wherein the heating/cooling unit is a thermoelectric unit.

10. The body thermal regulation system according to claim 1, wherein each of the at least one heating/cooling unit is positioned to individually heat and/or cool fluid circulating to each of the plurality of fluid-impervious compartments in the garment.

11. The body thermal regulation system according to claim 9, wherein said fluid circulation system comprises a plurality of fluid pumps, wherein each of the plurality of fluid pumps are positioned to individually draw fluid from each of the at least one heating/cooling unit and expel the heated or cooled fluid into each of the plurality of fluid-impervious compartments in the garment.

12. The body thermal regulation system according to claim 11, wherein said fluid pumps are positive displacement pumps.

13. The body thermal regulation system according to claim 1, further comprising:
at least one inlet manifold positioned to receive fluid from the plurality of fluid-impervious compartments in the garment; and
at least one outlet manifold positioned to receive fluid from the at least one heating/cooling unit.

14. The body thermal regulation system according to claim 13, further comprising:
a plurality of first temperature sensors positioned inside the at least one inlet manifold for measuring the temperature of fluid drawn from the plurality of fluid-impervious compartments in the garment; and
a plurality of second temperature sensors positioned inside the at least one outlet manifold for measuring the temperature of heated or cooled fluid from the at least one heating/cooling unit.

15. The body thermal regulation system according to claim 1, further comprising:
a plurality of third temperature sensors positioned to measure the temperature of fluid entering each of the plurality of fluid impervious compartments in the garment; and
a plurality of fourth temperature sensors positioned to measure the temperature of fluid exiting each of the plurality of fluid impervious compartments in the garment.

16. The body thermal regulation system according to claim 1, further comprising:
a plurality of fifth temperature sensors positioned to measure the temperature of the environment surrounding the body.

17. The body thermal regulation system according to claim 1, further comprising:
one or more flowmeters for measuring flow rate of fluid in the circulation system.

18. The body thermal regulation system according to claim 1, further comprising:
a phase change material for storing and releasing excess heat and cold.

19. The body thermal regulation system according to claim 1, further comprising:
an insulation layer positioned outside of and adjacent to said fluid-circulating garment.

20. A method for measuring heat fluxes in different parts of a body, said method comprising:
providing to a subject the body thermal regulation system according to claim 1;
measuring the temperatures of fluid entering and exiting each of the plurality of fluid-impervious compartments in the garment;
measuring the flow rates of fluid entering and exiting each of the plurality of fluid-impervious compartments in the garment; and
determining heat fluxes in different parts of the body.
